(19) United States
(12) Patent Application Publication
(10) Pub. No.: US 2010/0130904 A1
(43) Pub. Date: May 27, 2010

(54) METHOD TO INHIBIT PROLIFERATION AND GROWTH OF METASTASES

(75) Inventor: Michael T Kirber, Sharon, MA (US)

Correspondence Address: RONALD I. EISENSTEIN, 100 SUMMER STREET, NIXON PEABODY LLP, BOSTON, MA, 02110, US

(73) Assignee: TRUSTEES OF BOSTON UNIVERSITY, Boston, MA (US)

(21) Appl. No.: 11/995,958

(22) PCT Filed: Jul. 18, 2006
(86) PCT No.: PCT/US06/27746

§ 371 Date: Sep. 11, 2008

Related U.S. Application Data
(60) Provisional application No. 60/700,118, filed on Jul. 18, 2005.

(51) Int. Cl. A61M 1/36

(52) U.S. Cl. 604/5.01

(57) ABSTRACT

The present invention provides a method to reduce the amount of undesired growth factors in the circulating blood of a subject to prevent tumor growth and proliferation during or after a wound healing and/or other local tissue repair process on a subject, comprising extracorporeal adsorption of growth factors from blood of the subject and return of the treated blood to the subject.

### CROSS REFERENCE TO RELATED APPLICATIONS

The present application claims the benefit under 35 U.S.C. §119(e) of U.S. provisional application No. 60/700,118, filed Jul. 18, 2005, which application is herewith incorporated by reference in its entirety.

### BACKGROUND OF THE INVENTION

It is well known that surgical removal of a primary tumor is frequently followed by the rapid growth of multiple metastases and often death. When a subject is evaluated and metastases are found, removal of the primary lesion is often no longer considered the best course of treatment. Proliferation of metastases following removal of the primary lesion has led to the hypothesis that the primary lesion releases or causes the release of anti-angiogenic molecules, which block the growth of metastases. This has been an area of active research resulting in the identification of substances which may be useful in blocking tumor growth (for example, endostatin, angiostatin).

New forms of cancer treatment have involved blocking the effects of growth factors such as vascular endothelial growth factor (VEGF), fibroblast growth factor (FGF), platelet derived growth factor (PDGF), epidermal cell growth factor (EGF) and others. These agents can be receptor blockers such as monoclonal antibodies to the receptors (for example, HERCEPTIN™ (Trastuzumab), IMC-1121b), other competitive receptor-binding agents (for example, ERBITUX™ (cetuximab)). The agents can also block the activity of a growth factor receptor by interfering with its ability to function without necessarily blocking the extracellular binding site. For example, TARCEVA® (erlotinib) and IRESSA® (gefitinib) are thought to block the intracellular tyrosine kinase domain of the EGF receptor.

Following tissue injury caused by, for example, surgery or radiation therapy, circulating levels of the growth factors can become and are frequently elevated. This period of growth factor elevation during wound healing and/or inflammation may be a key factor in triggering the proliferation of metastases and micrometastases following surgery such as a surgery for treatment of cancer. However, blocking growth factors following surgery or radiation therapy could interfere with wound healing and consequently would not be a desirable course of treatment.

Thus, there is a need for methods to reduce systemic increase and circulation of growth factors in the body following surgery or other tissue injury without interfering with local surgical wound healing and/or repair of local tissue injury, such as injury caused by radiation therapy.

### SUMMARY OF THE INVENTION

The present invention provides a method to reduce the amount of undesired growth factors in the circulating blood of a subject to prevent tumor growth and proliferation during or after a wound healing and/or other local tissue repair process on a subject, comprising extracorporeal adsorption of growth factors from blood of the subject and return of the treated blood to the subject.

Any known means for extracorporeal removal can be used. For example, an apparatus for extracorporeal circulation of whole blood or plasma is connected to a subject. Growth factors can be removed from the blood or plasma in the apparatus by using a means for removing the growth factors such as affinity adsorption. One can use antibodies (including fragments thereof), growth factor receptors, nucleic acids, small molecules, molecules containing receptor or antibody mimetics, and the like to bind to the desired growth factor and remove that factor from the circulating blood or plasma. This systemic removal will not significantly affect the local levels of growth factors near a wound.

In one embodiment, the method is performed on a subject who is undergoing surgical removal of a tumor prior to administration of the methods of the present invention.

In one embodiment, the method is performed on a subject who underwent surgery unrelated to cancer prior to administration of the methods of the present invention.

In one embodiment, the method is performed on a subject who is at risk for cancer.

In one embodiment, the method is performed on a subject who is at risk for metastasis.

The present invention provides a method to prevent and/or inhibit metastatic and micrometastatic tumor growth in a subject undergoing surgical wound healing (i.e. treat a subject undergoing wound healing), comprising elimination of systemically circulating growth factors via extracorporeal adsorption of growth factors and return of blood to the subject.

In one embodiment, the surgical wound is a result of removal of a tumor.

### DETAILED DESCRIPTION OF THE INVENTION

The present invention is based on the discovery that the amount of growth factors can be reduced in systemic circulation without significantly altering local levels of growth factors near a site of a tissue injury, thereby preventing metastatic tumor growths following tissue injury, for example, surgery and radiation therapy.

The present invention provides a method to treat a subject by reducing the amount of undesired growth factors from the circulating blood of a subject, comprising extracorporeal adsorption of growth factors and return of blood to the subject.

In one embodiment, the method is performed in conjunction with a surgical removal of a tumor. In one embodiment, the method may be performed prior to surgery, during surgery, or after surgery.

In one embodiment at least 1, 2, 3, 4, 5 or more removal treatments are performed in conjunction with the surgical proceeding. One can begin this procedure at any time prior to surgery, for example, 24 hours, 8 hours, 5 hours, 4 hours, 3 hours, 2 hours, 1 hours, 0.5 hours or less. One can perform the procedure one or more times, or continuously during the surgery. One can also continue this procedure indefinitely. Typically, one performs one or more rounds of removal during a period of at least 8 hours, 24 hours, 36 hours, 48 hours, 72 hours and up to one week, 2 weeks, 3 weeks, 4 weeks, 5 weeks, 6 weeks, 7 weeks or even up to 2 months, 3 months, 4 months, 5 months or 6 months. One can begin the procedure following the surgery. Preferably one begins the treatment within at least one hour to one week of the surgery, for example within the first two days, preferably within the first day. All time periods in between are part of the procedure. In one preferred embodiment, treatment is completed during a 72 hour period from surgery. One does not have to use this method continually, but rather periodically to prevent build up of undesired circulating growth factors. Levels of growth factors can be determined by known means.

In one embodiment, the method is performed in conjunction with a surgery unrelated to cancer.

In one embodiment, the method is performed in conjunction with radiation therapy.

In one embodiment, the subject is at risk for cancer.

In one embodiment, the subject is at risk for metastasis.

The present invention provides a method to inhibit/prevent tumor growth, particularly metastasis and micrometastasis, in a subject undergoing surgical wound healing comprising, reduction or elimination of systemically circulating growth factors via extracorporeal adsorption of growth factors from blood and return of blood to the subject. In one embodiment, the surgical wound is a result of removal of a tumor.

In the methods of the present invention, one reduces the amount of circulating growth factors from the blood system using extracorporeal means. This is done by standard means, for example, an apparatus for extracorporeal circulation of whole blood or plasma is connected to the subject through tubing lines and blood access device(s). Such an apparatus should provide conduits for transporting the blood to an adsorption device and conduits for returning the processed blood or plasma to the subject. When plasma is processed through the adsorption device, a plasma separation device is typically used as well as means of mixing the concentrated blood with processed plasma. The later is normally achieved by leading the two components into an air-trap where the mixing occurs.

Devices for extracorporeal affinity adsorption have been developed by Aethlon Medical Inc. (San Diego, Calif.) for the removal of materials such as HIV and other viruses from blood (See U.S. Pat. No. 6,528,057 and U.S. Pat. App. No. 2204/0175291, which are incorporated herein in their entirety). Mitra Medical AB (Lund, Sweden) has developed an extracorporeal affinity adsorption device to filter labeled antibodies from the blood (See U.S. Pat. Nos. 6,723,318, 6,558,543, 6,251,394, 4,965,112, E.P. 0436717, Int'l Pat. App. Nos. WO 05/4615, WO 05/051424, WO 04/022111, WO 01/95857, and U.S. Pat. App. Nos. 2004/0052784, 2002/0159994, 2001/0023288, which are herein incorporated in their entirety). Other extracorporeal affinity adsorption devices have been disclosed in, for example, U.S. Pat. Nos. 4,714,556 and 4,787,974 to Ambrus and Csaba; 6,099,730 to Ameer et al.; and U.S. Pat. Nos. 6,039,946, 6,569,112, 6,676,622, and U.S. Pat. App. No. 2004/0220508 to Strahilevitz. Grovender et al. (Kidney International. (2004) 65:310-322) describe extracorporeal removal of β₂-microglobin using a single-chain antibody fragment-based immunoadsorbant device. The methods of the present invention contemplate use of the extracorporal affinity adsorption devices disclosed in the above publications and others known to the skilled artisan.

In one embodiment, one uses the method to reduce the amount of circulating growth factors other than cytokines. In one embodiment, one does not reduce the amount of TNF-α, IL-1β or IL-6. Extracorporeal techniques for blood clearance are widely used in kidney dialysis, where toxic materials build up in the blood due to the lack of kidney function. Other medical applications, in which an extracorporeal apparatus can be used, include: removal of radioactive materials; removal of toxic levels of metals, removal of toxins produced from bacteria or viruses; removal of toxic levels of drugs, and removal of whole cells (for example, cancerous cells, specific haematopoietic cells such as B, T, or NK cells) or removal of bacteria and viruses.

One can adapt the extracorporeal affinity adsorption device for the adsorption of the desired growth factor based upon the present specification. For example, the desired growth factors may be adsorbed through the use of antibodies, such as Fab', monoclonal antibodies, single chain antibodies, antibody fragments, etc., nucleic acids, small molecules, growth factor receptors, molecules containing receptor or antibody mimetics, any selective high affinity molecule or combinations thereof. The requirement is that such affinity compounds bind the growth factors that one wishes to reduce or remove from the circulation. Multiple species of affinity compounds may be used simultaneously in the methods of the invention. Single species of affinity compounds or multiple sets of species of affinity compounds may bind different growth factors.

The extracorporeal affinity device may contain a matrix (as in the device developed by Mitra Medical AB (Lund, Sweden)) that may be coated with a ligand, for example, an affinity compound or affinity compounds that bind growth factors. The extracorporeal affinity device may contain a compartment wherein the affinity compounds are contained (see, for example, U.S. Pat. No. 6,099,730). Here, the affinity compounds may be contained as immobilized on agarose, polyacrylamide or other composition of beads, or on micelles. The device may also comprise an ultrafiltration membrane wherein affinity compounds may be immobilized (see, for example, Aethlon Medical Inc. (San Diego, Calif.)). Other extracorporeal affinity adsorption devices also contain similar components on which affinity compounds (for example, bound to solid support) may be immobilized.

There is considerable evidence indicating that the overall process of tissue injury related to inflammation, healing and repair of damaged tissue (for example, wounds resulting from surgery or radiation therapy), including the necessary intercellular communication, is regulated in a coordinated manner in adult humans and other mammals by a number of specific soluble growth factors which are released within the wound environment. Such factors are released, for example, by degranulating platelets and incoming macrophages. These growth factors contribute to inducing neovascularisation, leucocyte chemotaxis, fibroblast proliferation, migration and deposition of collagen and other extracellular matrix molecules within the site of the injury. Growth factors that have been identified and isolated are generally specialized soluble proteins or polypeptides and include, but are not limited to, transforming growth factor alpha (TGF-α), transforming growth factor beta (TGF-β1, TGF-β2, TGF-β3 etc), platelet derived growth factor (PDGF), epidermal growth factor (EGF), insulin-like growth factors I and II (IGFI and IGFII) and acidic and basic fibroblast growth factors (acidic FGF and basic FGF). General reviews on growth factors can be found, for example, in articles by Mary H McGrath in Clinics in Plastic Surgery, Vol. 17, No. 3, July 1990, pp 421-432, and by George A Ksander in Annual Resorts in Medicinal Chemistry, 1989, Chap, 24 (published by Academic Press, Inc.) of which the contents are incorporated herein by reference. Growth factor activity is crucial for proper healing of injured tissue. However, increased systemic growth factor concentration in blood may result in harmful cell proliferation.

For example, many new cancers are initiated, and existing cancers and hyperproliferative disorders stimulated, by growth factors that affect either the cancer cell itself, or normal tissue around the cancer that facilitate survival of the cancer cell (for example, angiogenic factors). There is a direct correlation between the circulating level of certain growth factors and cancer proliferation. Stimulation by high levels of circulating growth factors released during a body's natural response to a tissue injury, such as surgical wound healing or radiation therapy, may increase a subject's risk for tumor growth and metastasis.

The present invention provides a method of preventing metastases by reducing the level of circulating growth factors in a subject to inhibit or prevent tumor growth and cancer recurrence, and to reduce or eliminate existing cancers, and metastatic and micrometastatic cell proliferation. Reduction of systemically circulating growth factors provides a novel way to prevent metastatic growth of secondary tumors following tissue injury caused by surgery or radiation therapy. Similarly, a subject who is at risk for cancer or at risk for recurrence of a cancer will benefit from the elimination of systemically circulating growth factors subsequent to surgery.

Reduction or removal of circulating growth factors by extracorporeal adsorption reduces systemic levels of circulating growth factors without significantly altering growth factor levels at the local site of tissue injury.

“Metastasis” and “micrometastis” refer to a focus of cancerous cells related to a preexisting cancer, referred to as primary tumor or cancer, but that developed remotely from this primary focus without continuity with it. The dissemination of these secondary foci typically takes place via lymphatic or hematic routes.

As used herein, the term “antibody” (Ab) refers to mammalian monoclonal antibodies, polyclonal antibodies, multispecific antibodies (for example, bispecific antibodies), antibody fragments, immunoglobulin chains or fragments thereof, such as Fv, Fab, Fab', F(ab')₂ or other antigen-binding sub-sequences of anti bodies, “single-chain Fv” antibody fragments or “diabodies”, so long as they exhibit the desired biological activity, for example, growth factor binding.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site.

Furthermore, in contrast to conventional (i.e. polyclonal) antibody preparations which typically include different antibodies directed against different determinants (epitopes), each monoclonal antibody is advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or made by recombinant DNA methods (see, for example, U.S. Pat. No. 4,816,567). The monoclonal antibodies may also be isolated from phage antibody libraries using the techniques described in, for example, Clackson et al., Nature, 352:624-628 (1991) and Marks et al., J. Mol. Biol., 222:581-597 (1991).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the reminder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)).

“Antibody fragments” comprise a portion of an intact antibody, generally the antigen-binding or variable region of the intact antibody. Examples of antibody fragments include Fab, Fab', F(ab')2, and Fv fragments, diabodies, single-chain antibody molecules, and multi-specific antibodies formed from antibody fragments.

“Single-chain Fv” antibody fragments comprise the VH and VL domains of antibody, wherein these domains are present in a single polypeptide chain. Generally, the Fv polypeptide further comprises a polypeptide linker: between the VH and VL domains which enables the sFv to form the desired structure for antigen binding. For a review of sFv, see Pluckthun in The Pharmacology of Monoclonal Antibodies, vol. 113, Rosenbourg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow paring between the two domains on the same chain, the domains are forced to pair with the complementary domains of another chain and create two antigen binding sites. Diabodies are described more fully in, for example, U.S. Pat. No. 5,591,828; WO 93/11161; and Hollinger et al., Proc. Natl. Acad. Sci. USA, 90:6444-6448 (1993).

As used herein, the term “affinity compound” includes any composition which binds specifically to a growth factor of the methods of the present invention. A binding composition or agent refers to a molecule that binds with specificity to the growth factor, for example, in a ligand-receptor type fashion or an antibody-antigen interaction, for example, proteins which specifically associate with the growth factor, for example, in a natural physiologically relevant protein-protein interaction, either covalent or non-covalent. The term “binding composition” includes small organic molecules, nucleic acids and polypeptides, such as a full antibody (preferably an isolated monoclonal human antibody) or antigen-binding fragment thereof. Antibodies and antigen binding fragments thereof, include, but are not limited to, monoclonal antibodies, polyclonal antibodies, bispecific antibodies, Fab antibody fragments, F(ab)2 antibody fragments, Fv antibody fragments (for example, VH or VL), single chain Fv antibody fragments and dsFv antibody fragments. In one embodiment, antibodies may be fully human antibodies or chimeric antibodies. Preferably, the antibody molecules are isolated monoclonal, fully human antibodies.

“Treatment” refers to both therapeutic treatment and prophylactic or preventative measures. Those in need of treatment include those already with the disorder as well as those in which the disorder is to be prevented.

“Subject” generally refers to a mammal. For purposes of treatment, mammal refers to any animal classified as a mammal, including humans, domestic and farm animals, and zoo, sports, or pet animals, such as dogs, horses, cats, cows, etc. Preferably, the mammal is human.

The terms “cancer” and “cancerous” refer to or describe the physiological condition in mammals that is typically characterized by unregulated cell growth and is intended to refer to both malignant and benign extreme or unregulated cell growth. Examples of cancer include, but are not limited to, carcinoma, lymphoma, blastoma, sarcoma, and leukemia. More particular examples of such cancers include squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, breast cancer, colon cancer, colorectal cancer, endometrial carcinoma, salivary gland carcinoma, kidney cancer, renal cancer, prostate cancer, vulval cancer, thyroid cancer, hepatic carcinoma and various types of head and neck cancer. The term “tumor” is used interchangeably with “cancer” herein.

“Growth factors” the removal/reduction of which is useful according to the methods of the present invention include growth factors, and polypeptide angiogenesis factors; and naturally modified derivatives and naturally occurring peptide fragments thereof. Growth factors induce or promote cell proliferation and/or angiogenesis. Growth factors contemplated by the invention comprise: Fibroblast Growth Factor (FGF) family members including, but not limited to, FGF-1/FGF acidic, FGF-2/FGF basic, FGF-3, FGF-4, FGF-5, FGF-6, FGF-7/KGF, FGF-8, FGF-9, FGF-10, FGF-11, FGF-12, FGF-12, FGF-13, FGF-14, FGF-15, FGF-16, FGF-17, FGF-18, FGF-19, FGF-20, FGF-21, FGF-22, FGF-23; Interleukins, including but not limited to, IL-1α, IL-1β, IL-2, IL-3, IL-4, IL-5, IL-6, IL-7, IL-8, IL-9, IL-10, IL-11, and IL-12; Colony Stimulating Factors including, but not limited to, Granulocyte Colony Stimulating Factor (G-CSF), Macrophage Colony Stimulating Factor (M-CSF or CSF-1), and GM-CSF; Epidermal Growth Factor (EGF) family members including, but not limited to, EGF, HB-EGF, Amphiregulin, Betacellulin, Epigen, Epiregulin, Neuregulin-3 (NRG-3), NRG1 isoform GGF2, NRG1 Isoform SMDF, NRG1-alpha/HRG1-alpha, NRG1-beta 1/HRG1-beta 1, TMEFF1, TMEFF2, and TGF-α; VEGF/PDGF family members, including, but not limited to, Vascular Endothelial Growth Factors (VEGF, otherwise known as Vascular Permeability Factor), VEGF, VEGF-B, VEGF-C, VEGF-D; Placental derived Growth Factors (PIGF), PIGF-1, PIGF-2, PIGF-3; Platelet-Derived Growth Factors (PDGF), PDGF, PDGF-A, PDGF-B, PDGF-C, PDGF-AB; Neuropilin-1, and Neuropilin-2; Transforming Growth Factor β (TGF-β) family members, including, but not limited to, TGF-β1, TGF-β2, TGF-β3, TGF-β4, and TGF-β5; Schwann cell-derived Growth Factor; Nerve Growth Factor (NGF); Insulin-like Growth Factors 1 and 2 (IGF-1 and IGF-2); Glial Growth Factor; Tumor Necrosis Factors TNF-α and TNF-β; Connective tissue growth factor (CTGF/CCN2); NOV/CCN3; PD-ECGF/gliostatin; Endocrine Gland-derived Vascular Endothelial Growth Factor/prokineticin-1 (EG-VEGF/PK1); Hepassocin; Hepatocyte Growth Factor (HGF/hepapoietin A/scatter factor); Beta subunit of Nerve Growth Factor (β-NGF); Progranulin; Thrombopoietin; Prolactin; Prostaglandins; and Growth hormone (GH1 and GH2).

The method of the present invention uses known technologies to immobilize enzymes, chelators, and antibodies in dialysis-like cartridges has been developed (see, for example, Ambrus et al. Science 201(4358): 837-839, 1978; Ambrus et al. Ann Intern Med 106(4): 531-537, 1987; Kalghatgi et al. Res Commun Chem Pathol Pharmacol 27(3): 551-561, 1980) and is incorporated herein by reference. An illustration of preparing proteins for immobilization to the device developed, for example, by Aethlon Medical Inc. is presented, for example, in U.S. Pat. Nos. 4,714,556 and 4,787,974, 5,528,057. Similar technologies can be used in the present invention.

For example, for binding of affinity molecules to the ultrafiltration membrane, matrix or other solid support, the polymers of the solid support are first activated, i.e., made susceptible for combining chemically with proteins, by using processes known in the art. Any number of different polymers can be used. To obtain a reactive polyacrylic acid polymer, for example, carbodiimides can be used (Valuev et al., 1998, Biomaterials, 19:41-3.). Once the polymer has been activated, the affinity molecules can be attached directly or via a linker. Suitable linkers include, but are not limited to, avidin, streptavidin, biotin, protein A, and protein G. For example, antibodies to specific growth factors may be bound to streptavidin coated polymers of the ultrafiltration membrane. The streptavidin coated ultrafiltration membrane can also be used for the attachment of oligonucleotide to which a biotin labeled base has been added to the 3′ end. The antibodies may also be directly bound to the polymer of the ultrafiltration membrane using coupling agents such as bifunctional reagents, or may be indirectly bound. For example, Protein A or Protein G may be used to immobilize IgG against specific growth factors.

For affinity absorbents, the solid support may be of various shapes and chemical compositions. It may, for example, constitute a column house filled with particulate polymers, the latter of natural origin or artificially made. The particles may be macroporous or their surface may be grafted, the latter in order to enlarge the surface area. The particles may be spherical or granulated and be based on polysaccharides, ceramic material, glass, silica, plastic, or any combination of these or a like material. A combination of these could, for example, be solid particles coated with a suitable polymer of natural origin or artificially made. Artificial membranes may also be used. These may be flat sheet membranes made of cellulose, polyamide, polysulfone, polypropylene or other types of material which are sufficiently inert, biocompatible, non-toxic and to which the receptor could be immobilized, either directly or after chemical modification of the membrane surface. Capillary membranes, like the hollow fibers made from cellulose, polypropylene or other materials suitable for this type of membranes, may also be used.

In another embodiment the solid support is coated by ligands which exhibit a specific interaction to the growth factor to be removed from the blood circulation. Such ligands can be chosen from a group comprising monoclonal antibodies including fragments or engineered counterparts thereof, aptamers, peptides, oligodeoxynucleosides including fragments thereof, intercalation reagents including dyestuffs, oligosaccharides and chelating groups interacting with the growth factor to be removed.

In another embodiment the adsorption device contains an immobilized receptor binding specifically to the growth factor on the solid support. Alternatively, the region of the receptor, that is the peptide fragment, that is the site of binding for the growth factor, may be used as an affinity compound. Any type of affinity ligand/immobilized receptor combinations such as “antibodies and antigens/haptens” and “protein and co-factors” could be used in this application, provided that they exhibit a sufficiently high binding affinity and selectivity to the growth factors and that the ligand-receptor interaction is not interfered with by blood or other body fluids or tissues being in contact with the adsorption agent and/or the device.

In one embodiment, the affinity compounds bind to one or more growth factors including, but not limited to, FGF-1/FGF acidic, FGF-2/FGF basic, FGF-3, FGF-4, FGF-5, FGF-6, FGF-7/KGF, FGF-8, FGF-9, FGF-10, FGF-11, FGF-12, FGF-12, FGF-13, FGF-14, FGF-15, FGF-16, FGF-17, FGF-18, FGF-19, FGF-20, FGF-21, FGF-22, FGF-23, IL-1α, IL-1β, IL-2, IL-3, IL-4, IL-5, IL-6, IL-7, IL-8, IL-9, IL-10, IL-11, IL-12; G-CSF, M-CSF/CSF-1, GM-CSF, EGF, HB-EGF, Amphiregulin, Betacellulin, Epigen, Epiregulin, NRG-3, NRG1 isoform GGF2, NRG1 Isoform SMDF, NRG1-alpha/HRG1-alpha, NRG1-beta 1/HRG1-beta 1, TMEFF1, TMEFF2, TGF-α, VEGF, VEGF-B, VEGF-C, VEGF-D, PIGF-1, PIGF-2, PIGF-3, PDGF, PDGF-A, PDGF-B, PDGF-C, PDGF-AB, Neuropilin-1, Neuropilin-2, TGF-β1, TGF-β2, TGF-β3, TGF-β4, TGF-β5, Schwann cell-derived Growth Factor, NGF, IGF-1 and IGF-2, Glial Growth Factor, TNF-α, TNF-β, CTGF/CCN2, NOVACCN3, PD-ECGF/gliostatin, EG-VEGF/PK1, Hepassocin, HGF/hepapoietin A/scatter factor, β-NGF, Progranulin, Thrombopoietin, Prolactin, Prostaglandins, GH1, GH2 and any combination thereof. In a preferred embodiment, the affinity compounds bind to growth factors comprising TGF-α, TGF-β1, TGF-β2, TGF-β3, PDGF, EGF, IGF-1, IGF-2, FGF-1/FGF acidic, FGF-2/FGF basic, VEGF, TNF-α, FGF-7/KGF and any combination thereof.

The anti-growth factor antibodies that can be used in the methods of the present invention are commercially available from, for example, R&D Systems (Minneapolis, Minn.), Abcam Limited (Cambridge, UK), Sigma-Aldrich (St. Louis, Mo.) and Upstate Biotechnology (Charlottesville, Va.). Alternatively, growth factor antibodies may be produced by methods well known to those skilled in the art. For example, monoclonal antibodies to growth factor (preferably mammalian; more preferably human) can be produced by generation of hybridomas in accordance with known methods. Hybridomas formed in this manner are then screened using standard methods, such as ELISA, to identify one or more hybridomas that produce an antibody that specifically binds to a particular growth factor. Full-length growth factors may be used as the immunogen, or, alternatively, antigenic peptide fragments of growth factors may be used.

As an alternative to preparing monoclonal antibody-secreting hybridomas, a monoclonal antibody to a growth factor may be identified and isolated by screening a recombinant combinatorial immunoglobulin library (for example, an antibody phage display library) to thereby isolate immunoglobulin library members that bind to the growth factor. Kits for generating and screening phage display libraries are commercially available from, for example, Dyax Corp. (Cambridge, Mass.) and Maxim Biotech (South San Francisco, Calif.). Additionally, examples of methods and reagents particularly amenable for use in generating and screening antibody display libraries can be found in the literature.

Polyclonal sera and antibodies may be produced by immunizing a suitable subject, such as a rabbit, with a growth factor (preferably mammalian; more preferably human) or an antigenic fragment thereof. The antibody titer in the immunized subject may be monitored over time by standard techniques, such as with ELISA, using immobilized marker protein. If desired, the antibody molecules directed the growth factor may be isolated from the subject or culture media and further purified by well-known techniques, such as protein A chromatography, to obtain an IgG fraction.

Fragments of antibodies to a growth factor may be produced by cleavage of the antibodies in accordance with methods well known in the art. For example, immunologically active F(ab') and F(ab')₂ fragments may be generated by treating the antibodies with an enzyme such as pepsin. Additionally, chimeric, humanized, and single-chain antibodies to a growth factor, comprising both human and nonhuman portions, may be produced using standard recombinant DNA techniques. Humanized antibodies to a growth factor may also be produced using transgenic mice that are incapable of expressing endogenous immunoglobulin heavy and light chain genes, but which can express human heavy and light chain genes.

The subject treated by the methods of the present invention may be a subject who is undergoing surgical wound healing. In one embodiment, the surgery is for removal of a tumor. In one embodiment, the tumor is a primary tumor. In one embodiment, the subject is at risk for a tumor, such as a subject identified as carrying tumor susceptibility mutations in genes such as BRCA1, BRCA2, HPC1, MLH1, or MSH2. In one embodiment, the subject is at risk for recurrence of cancer, for example, the subject was treated for a cancer prior to infliction of the wound and is at risk for recurrence of that cancer. In one embodiment, the surgery is unrelated to tumor treatment. In one embodiment, the subject is undergoing wound healing not induced by surgery, such as trauma (for example, a broken bone, local burn, etc). For example, a subject carrying a BRCA2 mutation is treated for injuries sustained in a car accident concurrently with the methods of the present invention.

Accordingly, the invention provides a method for reducing the amount of at least one circulating growth factor from circulating blood of a subject with a tissue injury, comprising contacting at least a portion of the circulating blood of a subject affected with a tissue injury with an extracorporeal adsorption device wherein the device comprises at least one adsorption compound that binds to at least one growth factor in the circulating blood of the subject. In one embodiment, least one growth factor is selected from the group consisting of TGF-α, TGF-β1, TGF-β2, TGF-β3, PDGF, EGF, IGF-1, IGF-2, FGF-1, FGF-2 (basic FGF), VEGF, TNF-α, FGF-7 and any combination thereof.

In one embodiment, the tissue injury is caused by surgery. For example, the tissue injury can be induced during removal of a tumor or caused by radiation therapy.

In one embodiment, the subject has or is at risk for developing cancer.

The invention also provides a method of treating a subject undergoing wound healing, comprising contacting the blood of said subject with an extracorporeal adsorption device wherein the device contains adsorption compounds that bind to growth factors in the subject's blood. The subject is preferably affected with a surgical wound. The surgical wound is preferably induced during surgical removal of a tumor. In one embodiment, the subject with the tissue injury is at risk for cancer. In one embodiment, the growth factors are selected from the group consisting of FGF-1/FGF acidic, FGF-2/FGF basic, FGF-3, FGF-4, FGF-5, FGF-6, FGF-7/KGF, FGF-8, FGF-9, FGF-10, FGF-11, FGF-12, FGF-12, FGF-13, FGF-14, FGF-15, FGF-16, FGF-17, FGF-18, FGF-19, FGF-20, FGF-21, FGF-22, FGF-23, IL-1α, IL-1β, IL-2, IL-3, IL-4, IL-5, IL-6, IL-7, IL-8, IL-9, IL-10, IL-11, IL-12; G-CSF, M-CSF/CSF-1, GM-CSF, EGF, HB-EGF, Amphiregulin, Betacellulin, Epigen, Epiregulin, NRG-3, NRG1 isoform GGF2, NRG1 Isoform SMDF, NRG1-alpha/HRG1-alpha, NRG1-beta 1/HRG1-beta 1, TMEFF1, TMEFF2, TGF-α, VEGF, VEGF-B, VEGF-C, VEGF-D, PIGF-1, PIGF-2, PIGF-3, PDGF, PDGF-A, PDGF-B, PDGF-C, PDGF-AB, Neuropilin-1, Neuropilin-2, TGF-β1, TGF-β2, TGF-β3, TGF-β4, TGF-β5, Schwann cell-derived Growth Factor, NGF, IGF-1 and IGF-2, Glial Growth Factor, TNF-α, TNF-β, CTGF/CCN2, NOV/CCN3, PD-ECGF/gliostatin, EG-VEGF/PK1, Hepassocin, HGF/hepapoietin A/scatter factor, β-NGF, Progranulin, Thrombopoietin, Prolactin, Prostaglandins, GH1, GH2, and any combination thereof. In one embodiment, the growth factor is selected from the group consisting of TGF-α, TGF-1β, TGF-β2, TGF-β3, PDGF, EGF, IGF-1, IGF-2, FGF-1, FGF-2 (basic FGF), VEGF, TNF-α, FGF-7 and any combination thereof.

Alternatively, the growth factors are selected from the group consisting of TGF-α, TGF-β1, TGF-β2, TGF-β3, PDGF, EGF, IGF-1, IGF-2, FGF-1/FGF acidic, FGF-2/FGF basic, VEGF, TNF-α, FGF-7/KGF, and any combination thereof.

The invention further provides a method of treating an individual undergoing removal of a cancer comprising removing undesired circulating growth factors from the individual. The circulating growth factors are preferably removed by an extracorporeal adsorption device. The device preferably comprises at least one adsorption compound that binds to at least one growth factor in the circulating blood of the individual.

In one preferred embodiment, the undesired circulating growth factor is selected from the group consisting of TGF-α, TGF-β1, TGF-β2, TGF-β3, PDGF, EGF, IGF-1, IGF-2, FGF-1, FGF-2, VEGF, TNF-α, FGF-7 and any combination thereof. In an alternative embodiment, the growth factor is selected from the group consisting of TGF-α, TGF-β1, TGF-β2, TGF-β3, PDGF, EGF, IGF-1, IGF-2, FGF-1, FGF-2 (basic FGF), VEGF, TNF-α, FGF-7 and any combination thereof.

The invention also provides for the use of an extracorporeal device to remove at least one undesired circulating growth factor from the blood of an individual with a tissue injury, such as tissue injury icaused by surgery or radiation therapy. In one preferred embodiment, the subject is at risk for cancer. In another embodiment, the subject has or has had a cancer, or a benign tumor. In one preferred embodiment, the undesired circulating growth factor is selected from the group consisting of TGF-α, TGF-β1, TGF-β2, TGF-β3, PDGF, EGF, IGF-1, IGF-2, FGF-1, FGF-2, VEGF including at least one VEGF sub type, TNF-α, FGF-7 and any combination thereof.

In conjunction of the methods of the present invention, the subject may also receive additional treatment modalities or therapeutics for the treatment of cancer or other condition. For example, the subject may undergo treatment for cancer including the taking of cytotoxic agents or chemotherapeutic agents simultaneously or concurrently with the methods of the present invention.

Cytotoxic agents include any substance that inhibits or prevents the function of cells and/or causes destruction of cells. Cytotoxic agents include radioactive isotopes, chemotherapeutic agents, and toxins such as, but not limited to, active toxins of bacterial, fungal, plant or animal origin, or fragments thereof. Some radionuclides, like indium-111, are used as diagnostic agents and are as such administered with low activity, but could also be used for therapeutic purposes if given in higher doses and are therefore also referred to as cytotoxic agents herein.

Chemotherapeutic agents are chemical compounds useful in the treatment of cancer. Examples of chemotherapeutic agents include Adriamycin, Doxorubicin, 5-Fluoruracil, Cytosine arabinoside (“Ara-C”), Cyclophosphamide, Thioptepa, Busulfan, Cytoxin, Taxol, Methotrexate, Cisplatin, Melphalan, Vinblastine, Bleomycin, Etoposide, Ifosfamide, Mitomycin C, Mitoxantrone, Vincristine, Vinorelbine, Carboplatin, Tenisposide, Duanomysin, Carminomycin, Aminopterin, Dactinomycin, Mitomycins, Esperamicins (see U.S. Pat. No. 4,675,187), Maytansinoids, Melphalan and other related nitrogen mustards.

In one embodiment, reduction of growth factors from systemic circulation means a reduction of at least about 5-10%, 10-25%, and in increasing preference, reductions of at least about 50%, 60%, 70%, 80%, 90% and 95%. Levels of circulating growth factor may be evaluated using any known technique, such as enzyme-linked immunosorbent assays (ELISA) or radioimmunoassays (RIA).

For the method of the present invention, blood is withdrawn from a subject and contacted with the extracorporeal adsorption device. Blood access may be achieved through peripheral vein catheters or, if higher blood flow is needed, through central vein catheters such as, but not limited to, subclavian or femoral catheters. The adsorption device can be directly perfused with blood from subjects and returned to the subjects without further manipulations. Alternatively, blood can be separated into plasma and cellular components by standard techniques. The plasma is then contacted with the adsorbent compounds to remove the growth factors by binding between growth factor and adsorbent compound. The plasma can then be recombined with the cellular components and returned to the subject. Alternatively, the cellular components may be returned to the subject separately. In one embodiment, at least 1, preferably 2, more preferably 3, 4, 5, 6 or even more volumes of blood are passed through the extracorporeal adsorption device. The treatment can be repeated periodically until a desired response has been achieved. For example, the treatment can be carried out for 4 hours once a week. Growth factor levels can be assessed in the effluent from the adsorption device by standard techniques such as ELISA and RIA.

All patents, patent applications and publications cited throughout the specification are incorporated herein by reference in their entirety.

1. A method for reducing the amount of at least one circulating growth factor from circulating blood of a subject with a tissue injury, comprising contacting at least a portion of the circulating blood of a subject affected with a tissue injury with an extracorporeal adsorption device wherein the device comprises at least one adsorption compound that binds to at least one growth factor in the circulating blood of the subject.

2. The method of claim 1, wherein the tissue injury is caused by surgery.

3. The method of claim 2, wherein the surgical wound is induced during removal of a tumor.

4. The method of claim 1, wherein the tissue injury is caused by radiation therapy.

5. The method of claim 1, wherein the subject has or is at risk for developing cancer.

6. The method of claim 1, wherein at least one growth factor is selected from the group consisting of TGF-α, TGF-β1, TGF-β2, TGF-β3, PDGF, EGF, IGF-1, IGF-2, FGF-1, FGF-2 basic, VEGF, TNF-α, FGF-7 and any combination thereof.

7. A method of treating a subject undergoing wound healing, comprising contacting the blood of said subject with an extracorporeal adsorption device wherein the device contains adsorption compounds that bind to growth factors in the subject's blood.

8. The method of claim 7, wherein the subject is affected with a surgical wound.

9. The method of claim 7, wherein the subject is affected with a surgical wound induced during surgical removal of a tumor.

10. The method of claim 7, wherein the subject is at risk for cancer.

11. The method of claim 7, wherein the growth factors are selected from the group consisting of FGF-1/FGF acidic, FGF-2/FGF basic, FGF-3, FGF-4, FGF-5, FGF-6, FGF-7/KGF, FGF-8, FGF-9, FGF-10, FGF-11, FGF-12, FGF-12, FGF-13, FGF-14, FGF-15, FGF-16, FGF-17, FGF-18, FGF-19, FGF-20, FGF-21, FGF-22, FGF-23, IL-1α, IL-1β, IL-2, IL-3, IL-4, IL-5, IL-6, IL-7, IL-8, IL-9, IL-10, IL-11, IL-12; G-CSF, M-CSF/CSF-1, GM-CSF, EGF, HB-EGF, Amphiregulin, Betacellulin, Epigen, Epiregulin, NRG-3, NRG1 isoform GGF2, NRG1 Isoform SMDF, NRG1-alpha/HRG1-alpha, NRG1-beta 1/HRG1-beta 1, TMEFF1, TMEFF2, TGF-α, VEGF, VEGF-B, VEGF-C, VEGF-D, PIGF-1, PIGF-2, PIGF-3, PDGF, PDGF-A, PDGF-B, PDGF-C, PDGF-AB, Neuropilin-1, Neuropilin-2, TGF-β1, TGF-β2, TGF-β3, TGF-β4, TGF-β5, Schwann cell-derived Growth Factor, NGF, IGF-1 and IGF-2, Glial Growth Factor, TNF-α, TNF-β, CTGF/CCN2, NOV/CCN3, PD-ECGF/gliostatin, EG-VEGF/PK1, Hepassocin, HGF/hepapoietin A/scatter factor, β-NGF, Progranulin, Thrombopoietin, Prolactin, Prostaglandins, GH1, GH2, and any combination thereof.

12. The method of claim 7, wherein the growth factors are selected from the group consisting of TGF-a, TGF-β1, TGF-β2, TGF-β3, PDGF, EGF, IGF-1, IGF-2, FGF-1/FGF acidic, FGF-2/FGF basic, VEGF, TNF-α, FGF-7/KGF, and any combination thereof.

13. A method of treating an individual undergoing removal of a cancer comprising removing undesired circulating growth factors from the individual.

14. The method of claim 13, wherein the circulating growth factors are removed by an extracorporeal adsorption device.

15. The method of claim 14, wherein the device comprises at least one adsorption compound that binds to at least one growth factor in the circulating blood of the individual.

16. The method of claim 13, 14 or 15, wherein the undesired circulating growth factor is selected from the group consisting of TGF-α, TGF-β1, TGF-β2, TGF-β3, PDGF, EGF, IGF-1, IGF-2, FGF-1, FGF-2, VEGF, TNF-α, FGF-7 and any combination thereof.

17. The use of an extracorporeal device to remove at least one undesired circulating growth factor from the blood of an individual with a tissue injury.

18. The use of claim 17, wherein the tissue injury is caused by surgery or radiation therapy.

19. The use of claim 17 or 18, wherein the subject is at risk for cancer.

20. The use of claim 19, wherein the subject had a cancer.

21. The use of claim 17, 18, 19, or 20 wherein the undesired circulating growth factor is selected from the group consisting of TGF-α, TGF-β1, TGF-β2, TGF-β3, PDGF, EGF, IGF-1, IGF-2, FGF-1, FGF-2, VEGFF, TNF-α, FGF-7 and any combination thereof.
